(19) United States
(12) Patent Application Publication
(10) Pub. No.: US 2024/0263166 A1
(43) Pub. Date: Aug. 8, 2024

(54) Method of Purifying Nucleic Acids Using Modified Solid Phases

(71) Applicant: OMEGA BIO-TEK, INC., Norcross, GA (US)

(72) Inventors: Christopher Stuart, Brookhaven, GA (US); Daniel Flanigan, Atlanta, GA (US); Julie Baggs, Lawrenceville, GA (US)

(73) Assignee: OMEGA BIO-TEK, INC., Norcross, GA (US)

(21) Appl. No.: 18/429,891

(22) Filed: Feb. 1, 2024

Related U.S. Application Data
(60) Provisional application No. 63/482,897, filed on Feb. 2, 2023.

(51) Int. Cl. C12N 15/10

(52) U.S. Cl. CPC C12N 15/1013

(57) ABSTRACT

A method of purifying nucleic acids using solid phases that are modified by a blocking reagent to limit the binding area and/or binding site of the solid phases to reduce the binding capacity of the solid phase to the nucleic acids. The starting amount of the nucleic acids exceeds the binding capacity of the solid phase, and the amount of the purified nucleic acid is in a fix amount, not proportional to the starting amount of the nucleic acids.

### CROSS-REFERENCE TO RELATED APPLICATIONS

This application claims the benefit of U.S. Provisional Application No. 63/482,897 filed on Feb. 2, 2023, which is incorporated herein by reference in its entirety.

### FIELD AND BACKGROUND

The present disclosure relates to purifying nucleic acids with solid phases. Recent advancements in molecular techniques, including Next-Generation sequencing by Illumina, have led to increased importance and prevalence of nucleotide identification methods. DNA normalization is a crucial step in the Next Generation Sequencing (NGS) process, as it helps to ensure that the sequencing results are representative of the original sample. The goal of DNA normalization is to reduce the variability of sequencing results by equalizing the representation of DNA fragments in the final library. This is particularly important when sequencing complex samples, such as those from mixed populations or cancer samples, to ensure that the sequencing results accurately reflect the underlying biology of the sample. There are several methods for DNA normalization that can be used prior to NGS, including quantification of the nucleic acids and adjustment of the concentration with diluent, PCR-based methods. These methods are time consuming and error prone methods. The utilization of magnetically responsive particles for the automation of nucleic acid isolation has gained significant attention as a cost-effective and comprehensive solution in the field.

The exact mechanisms behind the adsorption of nucleic acids on mineral substrates in the presence of chaotropic reagents is not fully understood, but it is believed that this adsorption is caused by disruptions in the higher-order structures of the aqueous medium. This results in the adhesion or unfolding of dissolved nucleic acid molecules onto the surfaces of glass or silica-gel particles. When high levels of chaotropic salts are present, this adsorption process will typically occur almost completely. The adsorbed nucleic acids can be released by using buffers with low salt concentrations.

However, the strong binding affinity of nucleic acids to glass surfaces poses a significant challenge in their efficient removal. The utilization of porous silica-based magnetic particles, specifically designed for the reversible binding of nucleic acids, has been demonstrated to improve elution efficiency. However, the precise control of binding capacity of these porous silica-based magnetic particles remains a challenge in the field.

Western blots are a common technique used to detect specific proteins in a sample. In order to properly detect the protein of interest, blocking agents are often used to reduce non-specific binding. Common blocking agents include non-fat dry milk, bovine serum albumin (BSA), and gelatin. Non-fat dry milk is often used as a blocking agent due to its high protein content and low cost. BSA is also a popular blocking agent because it is a highly purified protein that is well tolerated by most primary and secondary antibodies. Gelatin is sometimes used as a blocking agent because it is a denatured form of collagen, a protein found in many cell membranes, thus it can be useful to block the non-specific binding of primary and secondary antibodies that may be directed against this protein.

### SUMMARY

The present disclosure relates to the field of nucleic acid purification, specifically the purification of RNA, DNA and PNA using surfaces modified with polymers or resins to control and fix the binding ability of the pourous silica surface. In certain embodiments, the present disclosure provides methods for reversibly binding at least one polynucleotide to a pourous microparticle surface that has been modified with a blocking agent. In certain embodiments, using polymer or resin with modification to modify magnetic bead surface by a polymer agent to block the pourous sites is provided. In certain embodiments, the polymer used is a polymeric polyol, specifically PVP. In other embodiments, the blocking agents are protein and/or chemically based, including but not limited to, BSA, spermidine, histadine, poly-his, casein, milk powder, hexylamine, polymeric or monomeric beta-glucan, and polyethylene glycols. In certain embodiments, the nucleic acid to be purified is a fragment of DNA prepared by digestion or ligation with an enzyme with varying polynucleotide length.

The methods described herein can be applied to any number of polymers or resins to purify nucleic acids but adopts the process of using blocking agents with proteins to limit the surface area of microparticles and thus limit the binding ability of the magnetic microparticle.

Other systems, methods, features, and advantages of the present disclosure will be or become apparent to one with skill in the art upon examination of the drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the drawings attached with the present disclosure. The components in the drawings are not necessarily to scale, emphasis instead being placed upon clearly illustrating the principles of the present disclosure. Moreover, in the drawings, like reference numerals designate corresponding parts throughout the several views.

FIG. 1A-1B. Comparison of purification of 500 BP nucleic acid fragment using unblocked (1A) and blocked (1B) pourous magnetic beads. FIG. 1A shows that unblocked pourous magnetic beads return a various amount of nucleic acid in relation to the input amount of nucleic acid. FIG. 1B shows that blocking reagent with pourous magnetic beads allows for fixed amount of nucleic acid to bind to the magnetic beads. The binding capacity of the magnetic beads is reached and is determined by how many magnetic beads are added to/used in the reaction.

FIG. 2A-2B. Purification of 200 BP (2A) and 1000 BP (2B) nucleic acid fragment using blocked pourous magnetic beads. They illustrate that blocking allows normalization with low % Differences and Coefficient Variation in normalized groups (200-1000 ng).

FIGS. 3A-3D. illustrate the relationship between the quantity of the blocked pourous magnetic beads and the binding capacity. FIG. 3A illustrates that varying blocked magnetic beads quantity changes the fixed binding capacity. Using more blocked magnetic beads requires more DNA to reach the critical level of 100 ng/uL beads.” to reach a critical level. FIG. 3B illustrates more blocked magnetic beads require more DNAs to be bound to. FIG. 3C illustrates that DNA return is linear with the amount of blocked magnetic beads used for purification. FIG. 3D illustrates that same amount of DNA is bound per unit of blocked magnetic beads.

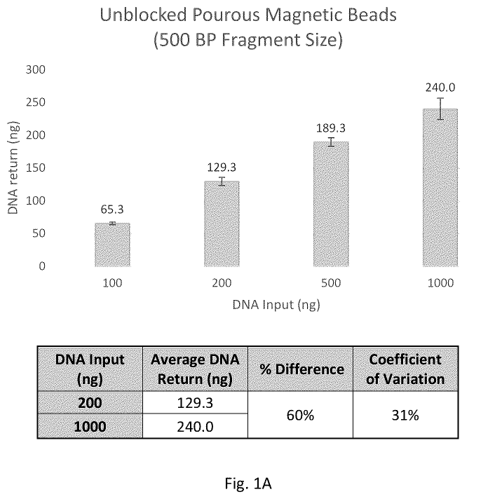

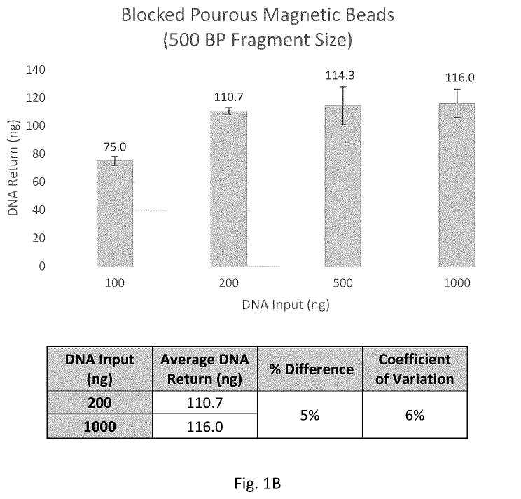

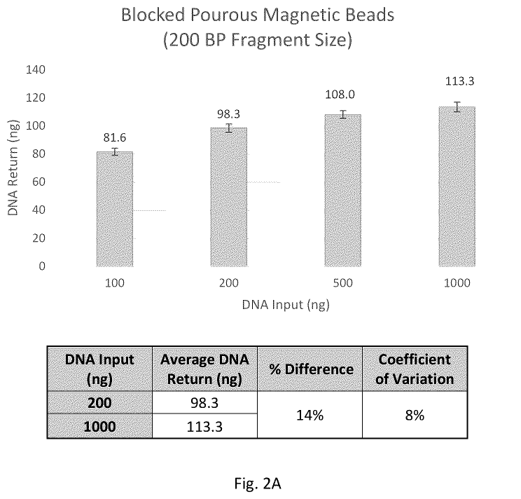

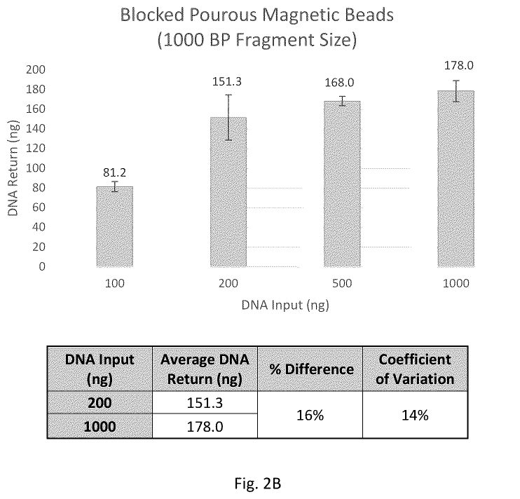

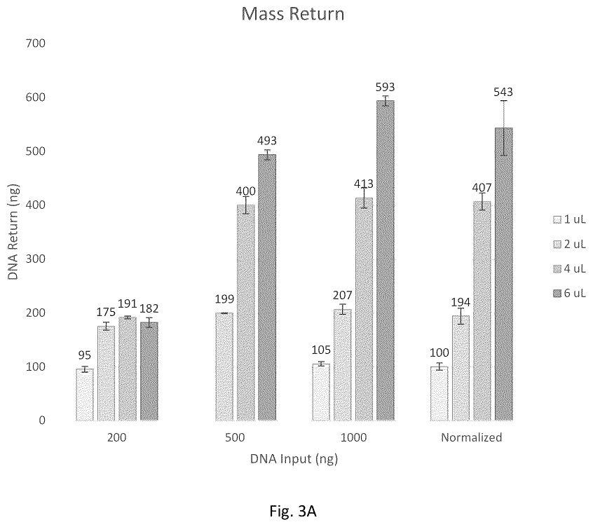

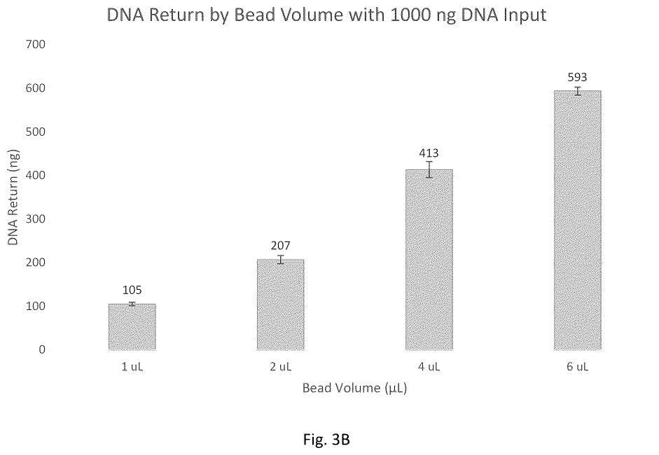

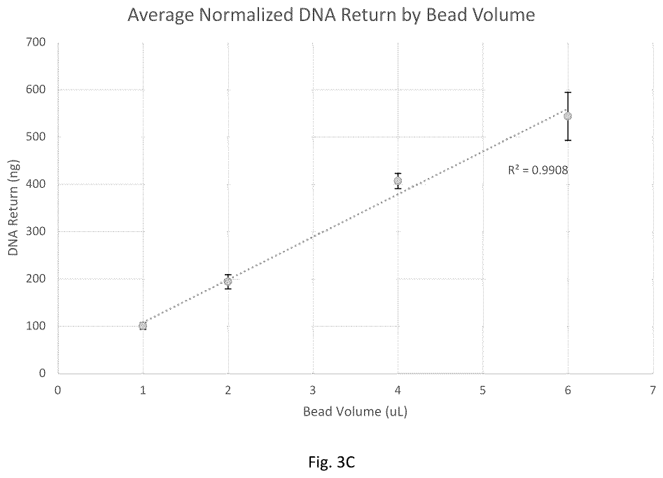

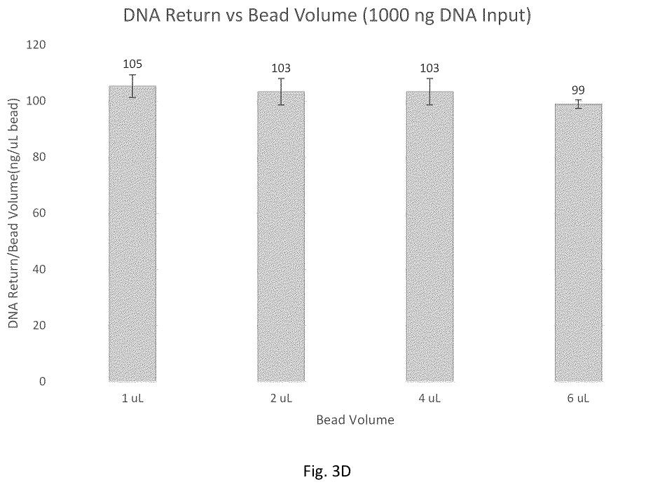

### DETAILED DESCRIPTION

The present disclosure provides a method of purifying nucleic acids using magnetic beads modified with a blocking agent to limit their surface area and/or lower the binding capacity of the porous magnetic beads and the amount/number of binding sites for nucleic acid. The total amount of nucleic acid output can be changed by modifying the amount/number of the magnetic beads. To remove contaminants, the nucleic acid-bead complex is washed, and the nucleic acids are released in a low solution or water. However, the amount of nucleic acid eluted from the magnetic beads is fixed and not in proportion to the input amount.

Additional advantages of the present disclosure will be set forth in part in the description which follows, and in part will be obvious from the description, or can be learned by practice of the present disclosure. The advantages of the present disclosure will be realized and attained by means of the elements and combinations particularly pointed out in the appended claims. It is to be understood that both the foregoing general description and the following detailed description are exemplary and explanatory only and are not restrictive of the invention, as claimed.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “a metal,” or “a substrate,” includes, but are not limited to, mixtures or combinations of two or more such catalysts, metals, or substrates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Unless otherwise specified, temperatures referred to herein are based on atmospheric pressure (i.e., one atmosphere).

The present disclosure provides a method of purifying nucleic acids using solid phases of magnetic particles or beads modified with a blocking reagent thus limiting the surface area of the magnetic particles or beads and binding sites to lower the binding capacity of the magnetic particles or beads. The bound nucleic acids are washed to remove any residual contaminants and then eluted from the magnetic particles or beads in low or no salt solution. The amount of output nucleic acids can be adjusted by changing the size of the nucleic acid and/or the amount of modified magnetic particles or beads added. The input amount of the nucleic acids does not change the output amount of the nucleic acids, once the binding capacity of the modified magnetic beads has been exceeded.

In certain embodiments, the blocking reagent is the polymer, such as polymeric polyol, specifically Polyvinylpyrrolidone (PVP), In other embodiments, the blocking reagents are protein and protein derivates. In yet other embodiments, the blocking reagents are chemically based blocking agents, including but not limited to, BSA, succinic anhydride, spermidine, histadine, poly-his, casein, milk powder, hexylamine, polymeric or monomeric beta-glucan, and polyethylene glycols.

In certain embodiments, the purification method provides that the nucleic acids are bound to solid phases of magnetic particles or beads. In certain embodiments, the magnetic particles or beads contain a pourous surface. In certain embodiments, the magnetic particles or beads are subjected to the blocking reagent prior to the reaction or during the binding of the nucleic acids to the magnetic particles or beads. In certain embodiments, the amount and the length of time the magnetic particles or beads modified with the blocking reagents can be adjusted to increase or decrease amounts of nucleic acids bound to the magnetic particles or beads.

In certain embodiments, nucleic acids bind to the modified magnetic particles or beads in a solution comprising one or more salts, including but not limited to, sodium chloride, magnesium chloride, calcium chloride, potassium chloride, lithium chloride, barium chloride, cesium chloride, sodium perchlorate, polyethylene glycol, guanidinium isothiocyanate, guanidinium hydrochloride, potassium iodide, and sodium iodide.

In certain embodiments, separation of the nucleic acids bound to the modified magnetic particles or beads is performed using a magnet.

In certain embodiments, the nucleic acids bound to at least one surface of the modified magnetic particles or beads are washed with a buffer solution that dissolves one or more impurities bound to the surface of the modified magnetic particles or beads while leaving only the nucleic acids bound thereon. In certain embodiments, the bound nucleic acids are then eluted in water, Tris-based buffer (TE buffer) solution or other buffer solutions containing low salts.

In certain embodiments, the purification method uses a polynucleotide solution that comprises a lysate of blood, a blood fraction, fresh tissue, fixed tissue, or an enzymatic reaction and/or a solution containing unpurified and/or purified nucleic acids. As used herein, nucleic acids refer to DNA, RNA, and PNA.

In certain embodiments, the purification method disclosed herein provides that the input amounts of nucleic acids exceed the binding capacity of the modified magnetic particles or beads. The output amounts of nucleic acids are not in proportion to the input amount of the nucleic acids and is a fixed amount.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The following describes exemplary blocking protocol and normalization protocol of the purifying method described herein.

The blocking protocol comprises the following steps:
1. Bring blocking agent to desired concentration in blocking buffer,
2. Add 80 uL blocking agent and 2 uL magnetic beads,
3. Shake beads on orbital shaker overnight at room temperature,
4. Wash beads 2× with CB buffer, and
5. Resuspend the magnetic beads in 82 uL CB Buffer.

The normalization protocol comprises the following steps:
1. Transfer 20 μL DNA to 96-well plate,
2. Add 82 uL bead mix to each well (from step 4),
3. Mix continuously at room temperate for 10 minutes,
4. Place on magnet, let sit at room temperate until beads completely magnetized,
5. Aspirate supernatant,
6. Remove plate from magnet,
7. Add 100 uL CB Buffer. Pipet up and down to mix thoroughly,
8. Place on magnet, let sit at room temperature until beads completely magnetized,
9. Aspirate and discard supernatant. Do not disturb beads,
10. Add 150 uL SPM Buffer. DO NOT remove plate from magnet,
11. Let sit at room temperature for 1 min,
12. Aspirate and discard supernatant. Do not disturb beads,
13. Repeat 10-12 for a second SPM wash,
14. Leave the plate on the magnet for 5 min to air dry, remove any residual liquid with a pipet,
15. Remove plate from magnet,
16. Add 20 uL elution buffer at 70° C. Pipet to mix thoroughly,
17. Mix continuously at room temperature for 5 min,
18. Place plate on magnet, let sit at room temperature until beads completely magnetized;
19. Transfer supernatant containing normalized DNA to a new plate; and
20. Store DNA at −20° C.

### REFERENCES

U.S. Pat. Nos. 4,233,169; 4,395,271; 4,297,337.
Proc., Et al., Under chaotropic or other high salt conditions, ie at high concentrations of chaotropic salt or another salt. Natl. Acad. USA 76 (1979) 615-619, Anal. Biochem. 121 (1982) 382-387], diatomeneenerde [Methods Enzymol. 65 (1979) 176-182] or natural silica [J. Clin. Microbiol. 28 (1990) 495-503, EP038963B1.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method of using a solid phase for purifying a nucleic acid from a sample, wherein said method comprises a step of modifying a surface of the solid phase with a blocking reagent to control and/or fix binding ability of the nucleic acid bound to the surface of the solid phase.

2. The method of claim 1, wherein the solid phase comprises magnetic beads or microparticles.

3. The method of claim 1, wherein the solid phase comprises a porous silica surface.

4. The method of claim 1, wherein the blocking reagent is selected from the group consisting of a polymer, a resin, a protein and/or protein derivative, and a chemical-based blocking reagent.

5. The method of claim 4, wherein the polymer is a polymeric polyol.

6. The method of claim 5, wherein the polymeric polyol is polyvinylpyrrolidone (PVP).

7. The method of claim 4, wherein the chemical-based blocking reagent is selected from the group consisting of BSA, succinic anhydride, spermidine, histadine, poly-his, casein, milk powder, hexylamine, polymeric or monomeric beta-glucan and polyethylene glycols.

8. The method of claim 1, wherein the surface of the solid phase is modified with the blocking reagent prior to or during binding of the nucleic acid to the surface of the solid phase.

9. The method of claim 8, wherein an amount of the solid phase being modified with the blocking agent is adjusted to increase or decrease an amount of the nucleic acids bound to the surface of the solid phase.

10. The method of claim 8, wherein a time duration of the surface of the solid phase being modified with the blocking agent is adjusted to increase or decrease an amount of the nucleic acids bound to the surface of the solid phase.

11. The method of claim 1, wherein the nucleic acid binds to the surface of the solid phase in a solution comprising one or more salts.

12. The method of claim 11, wherein the salt is selected from the group consisting of sodium chloride, magnesium chloride, calcium chloride, potassium chloride, lithium chloride, barium chloride, cesium chloride, sodium perchlorate, polyethylene glycol, guanidinium isothiocyanate, guanidinium hydrochloride, potassium iodide, and sodium iodide.

13. The method of claim 2, wherein the nucleic acid bound to the surface of the magnetic beads or microparticles is separated by a magnet.

14. The method of claim 1, wherein the nucleic acid bound to the surface of the solid phase is washed with a solution that dissolves one or more impurities bound to the solid phase but leaves the bound nucleic acid on the surface of the solid phase.

15. The method of claim 1, wherein the nucleic acid bound to the surface of the solid phase is eluded in water, Tris-based buffer (TE buffer) or a buffer comprising a low concentration of salt.

16. The method of claim 1, wherein an amount of an input nucleic acid exceeds the binding capacity of the nucleic acid bound to the surface of the solid phase.

17. The method of claim 16, wherein an amount of an output nucleic acid that is purified is in a fixed amount.

18. The method of claim 1, wherein the sample comprises a lysate of blood, a blood fraction, fresh tissue, fixed tissue, or an enzymatic reaction and/or a solution containing unpurified and/or purified nucleic acids.

19. The method of claim 1, wherein the nucleic acid is DNA, RNA, or PNA.

20. The method of claim 1, wherein the surface of the solid phase is modified by the blocking agent comprises the following steps:
a) providing the blocking agent to a desired concentration in a blocking buffer,
b) mixing a certain amount of the blocking agent with a certain amount of the solid phase,
c) shaking the mixture of step b) overnight at room temperature,
d) washing the solid phase from step c) with 2× washing buffer, and
e) resuspending the solid phase in a certain amount of washing buffer.
